(19) United States
(12) Patent Application Publication
(10) Pub. No.: US 2022/0361901 A1
(43) Pub. Date: Nov. 17, 2022

(54) PULMONARY EMBOLISM EXTRACTION DEVICE

(71) Applicant: TRIFORTIS LLC, Tucson, AZ (US)

(72) Inventors: Dexter De Leon, Tucson, AZ (US); Phat Le Tran, Tucson, AZ (US); David Moore, Tucson, AZ (US); Cody Mitts, Tucson, AZ (US)

(21) Appl. No.: 17/746,812

(22) Filed: May 17, 2022

Related U.S. Application Data
(60) Provisional application No. 63/189,331, filed on May 17, 2021.

(51) Int. Cl. A61B 17/22; A61M 1/34; A61M 1/36; A61M 60/279; A61M 60/37; A61M 60/13; A61M 60/562

(52) U.S. Cl. CPC A61B 17/22; A61M 1/34; A61M 1/3659; A61M 60/279; A61M 60/37; A61M 60/13; A61M 60/562; A61B 2017/22079

(57) ABSTRACT

A device for extracting arterial and pulmonary embolisms is described herein. The device comprises a suction catheter and a return catheter attached to a reservoir. The reservoir comprises two filters that filter out any unwanted material from the blood. The device may be controlled by a console with a pedal. Blood containing unwanted material is suctioned out of a patient, is filtered in the reservoir, and is returned to the patient. The device prevents blood loss from the patient by returning the blood back to the patient after it is filtered. Furthermore, the filtration system is designed to also remove air from the blood as it is suctioned from the patient.

### CROSS-REFERENCES TO RELATED APPLICATIONS

This application is a non-provisional and claims benefit of U.S. Provisional Application No. 63/189,331 filed May 17, 2021, the specification of which is incorporated herein in its entirety by reference.

### BACKGROUND OF THE INVENTION

The present invention relates to the removal of foreign material, in situ or embolic thrombus in the vascular tree like intracardiac thrombus and pulmonary embolism using the devices and methods described herein.

Pulmonary embolism is a blockage in the pulmonary arteries of the lungs. In most cases, pulmonary embolism is caused by blood clots that travel to the lungs from deep veins in the legs or, rarely, from veins in other parts of the body (deep vein thrombosis). Since the clots block blood flow to the lungs, pulmonary embolism can be life-threatening. However, prompt treatment greatly reduces the risk of death.

Current devices used for embolism extraction have outdated designs. One of these mechanical suction devices comprises a large catheter with a 60 mL syringe at the back to apply negative pressure for aspiration. The instruction for use includes: A) Floating a pigtail to the pulmonary artery; B) Delivering suction to aspirate the blood and the thrombus; and C) Discarding the aspirated blood. There are several issues with the use of this device. It is very time consuming and blood loss during the procedure is very significant. The pigtail is not designed to be floated to the pulmonary artery due to its stiffness and shape. Most operators use a wire and wire the right ventricle to the pulmonary artery, then pass the pigtail through the wire. This procedure is unsafe because the wire can pass through the tricuspid apparatus which can then be damaged as the catheter passes through the apparatus. The use of a 60 ml syringe to aspirate blood and clots and subsequently discarded can be detrimental due to blood loss. In some cases, up to about 1.5 L of blood can be aspirated, and after the procedure, the patient is anemic and hypotensive. Furthermore, the blood loss may then increase mortality. Multiple studies in cardiology have linked blood loss to poor outcomes.

In another mechanical suction system, a catheter is intended for use in procedures requiring the extracorporeal circulatory bypass support. Therefore, the aspirated blood may be re-infused simultaneously back to the patient. The system can minimize blood loss, but it still has many limitations that can generate adverse events. The system cannot handle air and the catheter cannot reach the pulmonary trunk. It is mainly used to aspirate materials in the right heart chambers. The system also requires a specialist to operate and aspirated materials cannot be seen readily for treatment assessment. In some cases, the filter can be clotted by the materials causing an exchange that can be detrimental to the patient. Additionally, crystalloid, therapeutics or blood products cannot be added with the device.

### BRIEF SUMMARY OF THE INVENTION

It is an objective of the present invention to provide devices and methods that allow for the aspiration of foreign bodies, clots, or infection material from a blood vessel and cardiac chambers, as specified in the independent claims. Embodiments of the invention are given in the dependent claims. Embodiments of the present invention can be freely combined with each other if they are not mutually exclusive.

The present invention features a device for aspirating blood from a patient. Blood is aspirated to remove a foreign body, a clot, or other infectious material from blood vessels and cardiac chambers. The aspirated blood is then filtered and transfused back to the patient. This process makes clot removal more efficient with no impact on blood loss. To prevent hemodynamic compromise, the volume of blood that is suctioned out is the same volume of blood being delivered back to the patient. The device may have a graded delivery of negative suction. As the clot is engaged, the pressure is increased. If blood flow is greater than 1 liter per minute, then pressure is decreased. The device comprises a reservoir that acts as a large filter and air trap. Air handling is an important part of the design as air should not be introduced into the patient's circulation. Furthermore, the device can be integrated to a cardiopulmonary bypass machine for hemodynamic support, or the device can also be used as a rapid transfuser of blood, therapeutics and crystalloid products.

One of the unique and inventive technical features of the present invention is the use of a long, flexible catheter that can reach the pulmonary artery. Current devices can place a catheter in the heart and are typically too rigid to place in the pulmonary artery. Another unique and inventive technical feature of the present invention is the automated application of negative pressure to aspirate blood/clot or any material from the patient. The device may have an automated graded delivery of negative pressure to provide ample negative suction while preventing hemodynamic compromise. As the blood is being aspirated from the patient, the blood is filtered in the reservoir of the device to remove/separate foreign bodies, clots, or other infectious material and also to remove bubbles and air, before the blood is transfused back to the patient. The conical shape of the funnel in the reservoir ensures that there is laminar flow of the blood as it is filtered, thus minimizing damage to the blood and eliminating the production of micro bubbles.

Without wishing to limit the invention to any theory or mechanism, it is believed that the technical features of the present invention advantageously ensure patient safety by preventing significant blood loss from the patient. The amount of blood that is aspirated from the patient is the same amount that is transfused back to the patient. Furthermore, the device described herein also eliminates air bubbles when filtering blood. None of the presently known prior references or work has the unique inventive technical features of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The patent application or application file contains at least one drawing executed in color. Copies of this patent or patent application publication with color drawings will be provided by the Office upon request and payment of the necessary fee.

The features and advantages of the present invention will become apparent from a consideration of the following detailed description presented in connection with the accompanying drawings in which:

FIG. 1 shows a schematic diagram of the embolism extraction device.

FIGS. 2A-2C show CAD drawings of the reservoir of the present invention. FIGS. 2A-2B show external views of the reservoir, and FIG. 2C shows the internal set-up of the reservoir, including the filtration system.

FIGS. 3A-3B show CAD drawings of the return catheter.

FIGS. 4A-4C show CAD drawings of the suction catheter.

FIG. 5A shows a CAD drawing of a console that may be connected to and that controls the system of the present invention. FIG. 5B shows a CAD drawing of a top view of a pedal that may be connected to the console to control the system. FIG. 5C shows a CAD drawing of a side view of the pedal.

FIGS. 6A-6C show CAD drawings of the daughter catheter.

FIGS. 7A-7C show complete CAD drawings of the suction catheter with a disk.

FIGS. 8A-8B shows flow charts illustrating operation of the console to stop or control flow to or from the reservoir.

FIG. 9 shows a CAD drawing of an airlock for sealing an end of a cannula of the present invention. As the screw cap engages the cannula, the flexible insert compresses and creates a seal around objects inserted into the cannula.

FIG. 10 shows a CAD drawing of a disk configured for deployment at the end of a catheter of the present invention. The inner or middle region of the catheter may be extended or retracted to deploy the disk.

FIG. 11 shows a schematic diagram of a manual extraction device of the present invention which uses a large lockable syringe embedded in a T-fashion between two duckbill one-way valves. When the syringe is pulled and locked, it will generate a negative suction to drain blood/clots/unwanted materials through the one-way valve. When it is pushed, the blood/unwanted materials will be pushed through the other one-way valve. The unwanted materials will be filtered and only the blood is to be returned to the patient. Multiple movable tubing clamps are in place to assist with priming the filter apparatus and to isolate the apparatus in the event of any parts that need to be exchanged. Multiple types of filter manifolds are illustrated.

### DETAILED DESCRIPTION OF THE INVENTION

Following is a list of elements corresponding to a particular element referred to herein:
100 embolism extraction device
120 first/suction catheter
122 first catheter first end
124 first catheter second end
130 reservoir
131 top chamber
132 first filter
33 middle compartment
134 second filter
136 bottom chamber
137 outlet
140 second/return catheter
142 second catheter first end
144 second catheter second end
200 console
210 console pedal

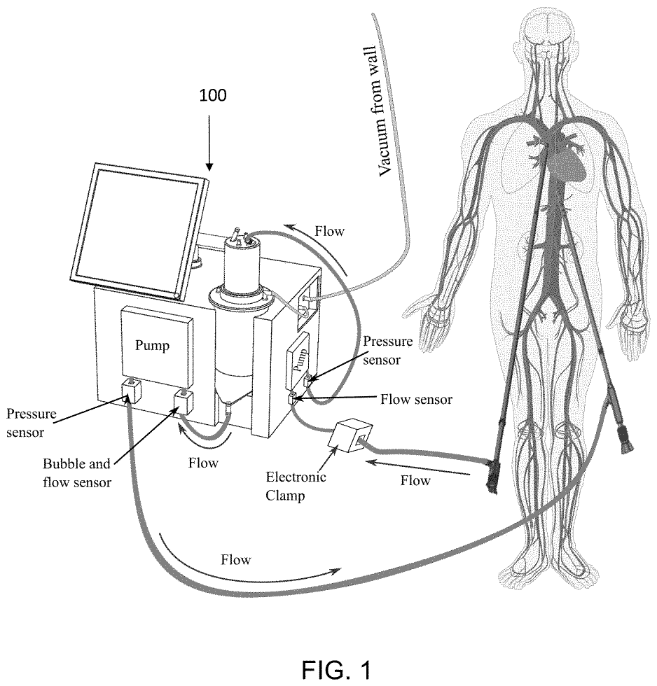

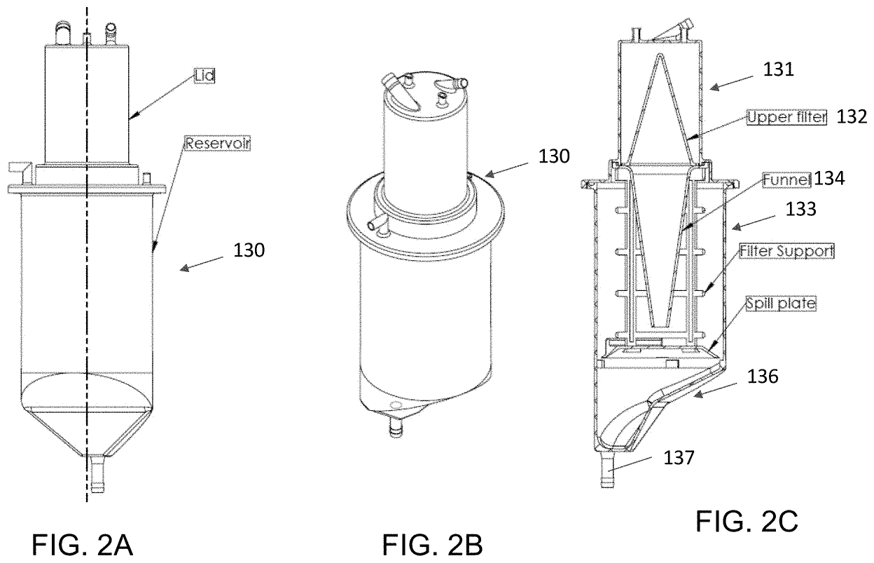

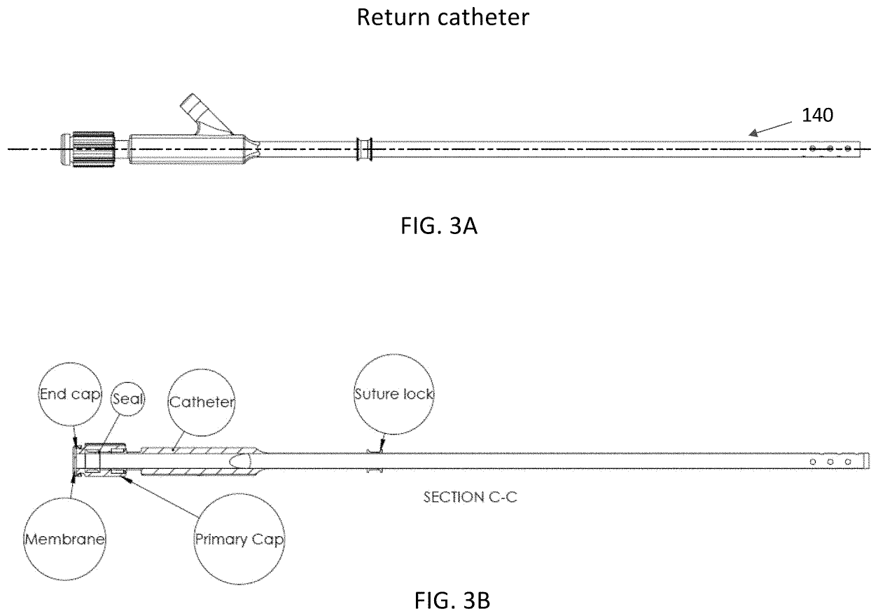

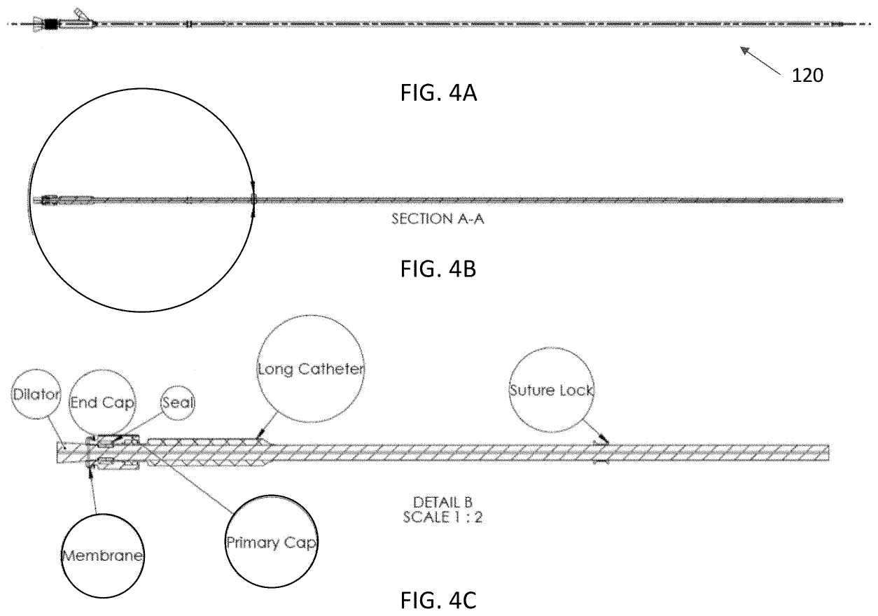

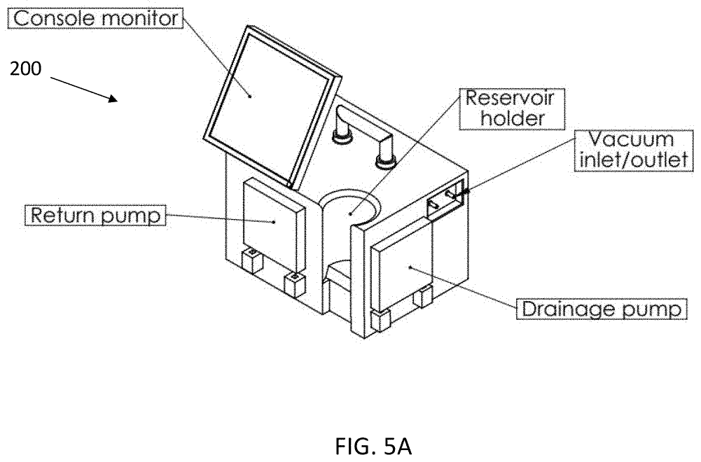

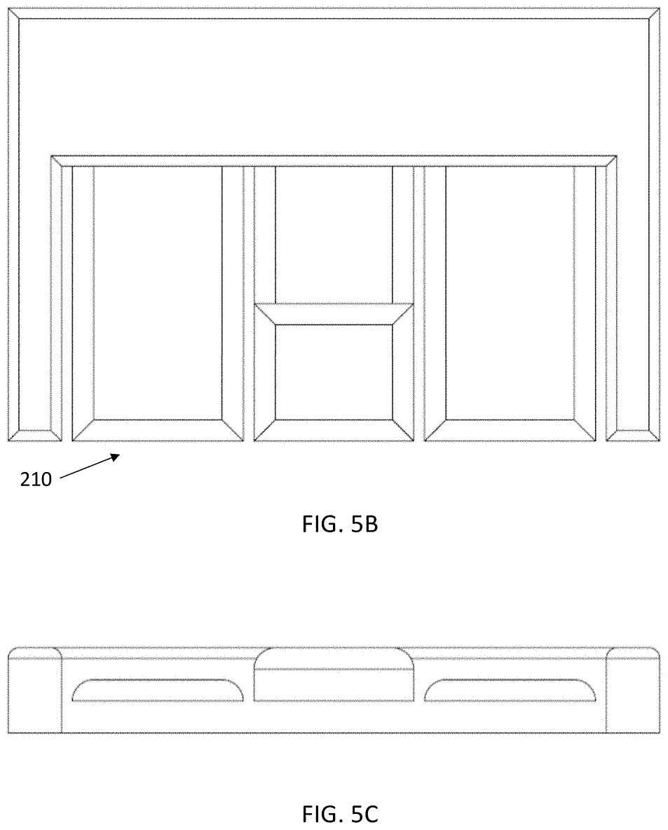

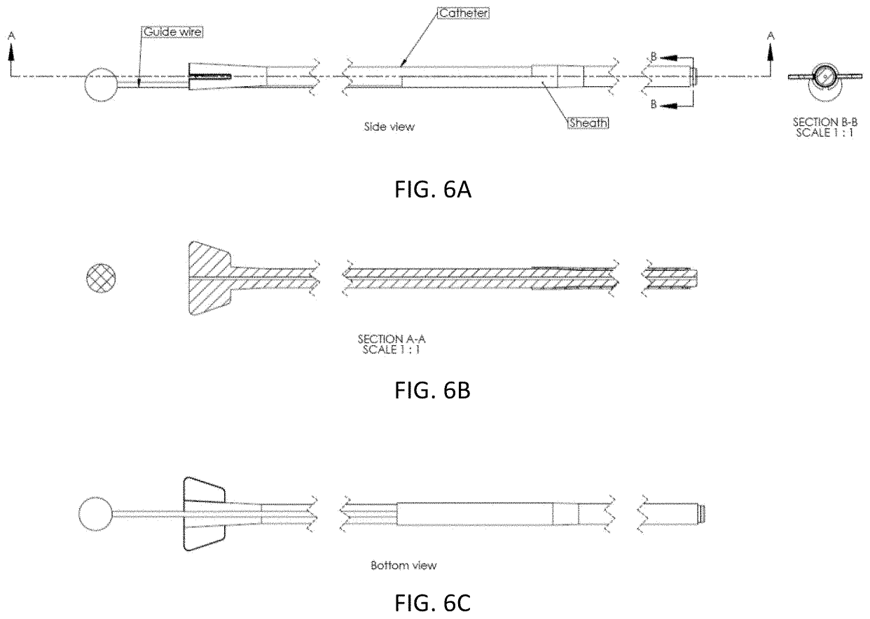

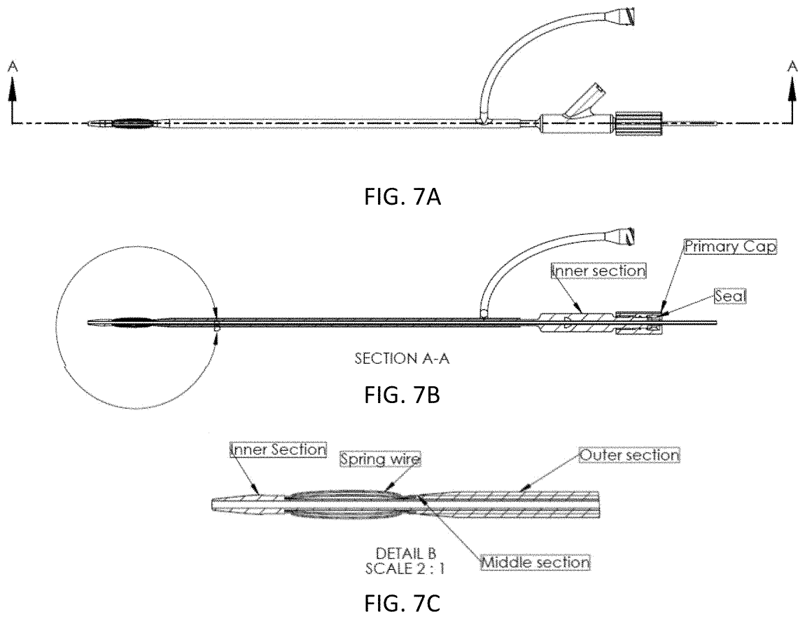

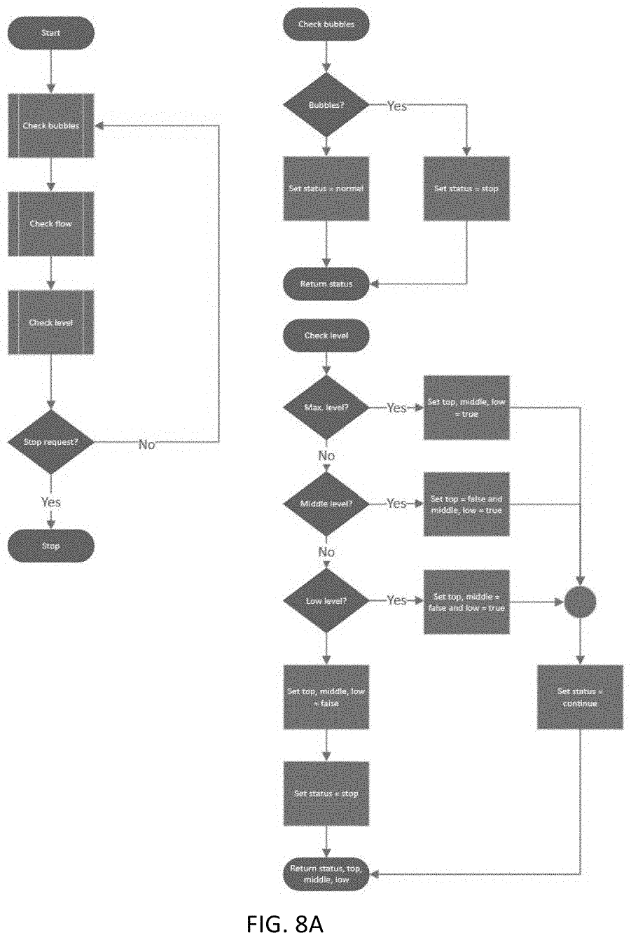

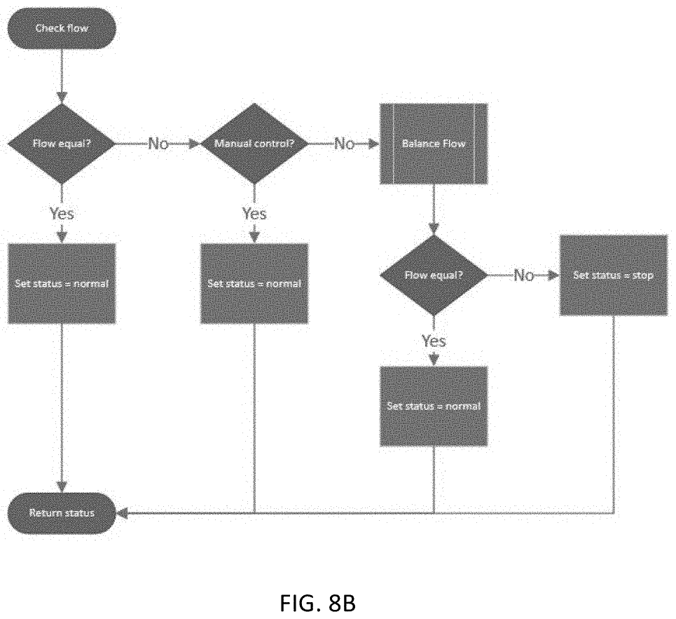

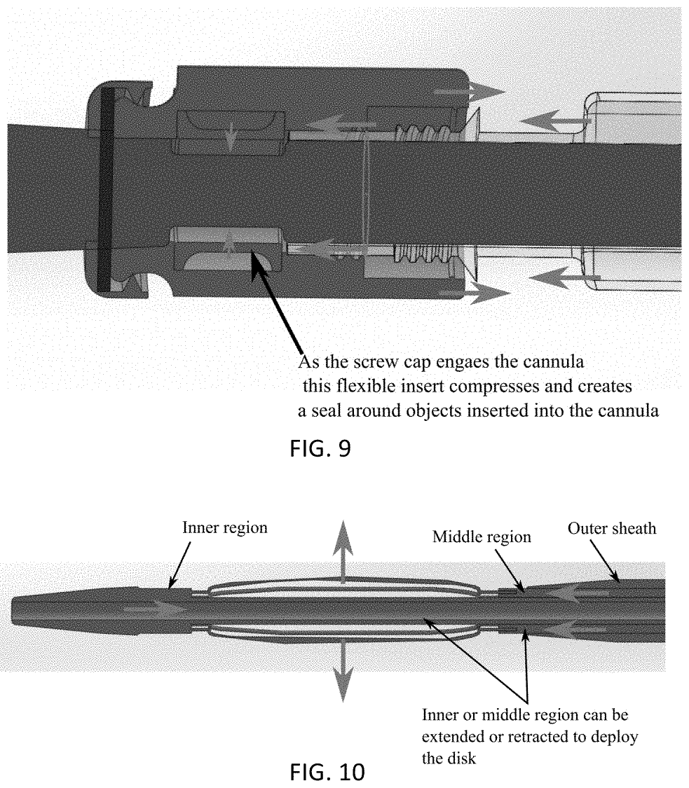

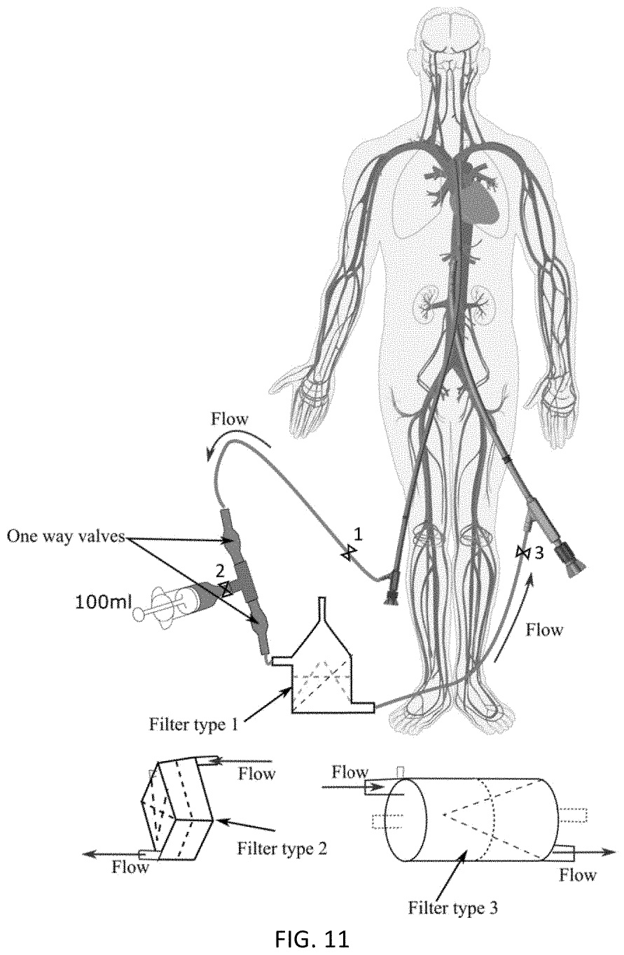

Referring now to FIG. 1, the present invention features a device (100) for extracting unwanted material from a blood vessel. In some embodiments, the device comprises a first tube (120), a reservoir (130), and a second tube (140). The first tube (120) may comprise a first end (122) that is configured to be disposed in a first blood vessel and a second end (124) that is connected to the reservoir (130). In some embodiments, the second end (124) may be slated into a roller pump or hospital central vacuum pump to mechanically generate negative pressure and drain the blood to the reservoir (130). The reservoir (130) may comprise a top chamber (131), a middle compartment (133), and a bottom chamber (136). In further embodiments, the top chamber (131) may have a first filter (132) disposed therein, and the middle compartment (133) may have a second filter (134) disposed therein. In some embodiments, the middle compartment (133) may be connected to a vacuum pump or a portable pump via a vacuum inlet (135). In other embodiments, the bottom chamber (136) comprises an outlet (137), and the bottom chamber is connected to a roller pump. In some embodiments, the second tube (140) comprises a first end (142) that is connected to the outlet (137) of the bottom chamber (136), and the second end (144) is configured to be disposed in a second blood vessel via a return cannula.

In some embodiments, the device (100) further comprises a console (200) that regulates the pressure of the vacuum pump and the roller pump. In preferred embodiments, the negative roller pump and vacuum pump controls flow of blood out of a patient and the other roller pump controls flow of blood into a patient. In other embodiments, the console (200) controls a pressure or a flow rate of the device. In other embodiments, the console further comprises a pedal (210) to control the pressure or the flowrate of the device. The negative pump may suction out blood from a patient using a negative pressure (e.g. between about −150 mmHg to −700 mmHg). In preferred embodiments, the pressure used to suction out the blood may be staggered. As a non-limiting example, blood may be continuously or cyclically suctioned from a patient at or up to −700 mmHg to engage the unwanted material. Once the unwanted material is suctioned out with the blood, the negative pressure may be decreased to limit the drainage. Non-limiting examples of the unwanted material include foreign bodies, clots, or other infectious material. In other embodiments, the console (200) is configured to stop negative suction or drainage if a patient's mean arterial blood pressure drops below 65 mmHg. Meanwhile, the console will continue to return blood or crystalloid to restore hemodynamics.

In another preferred embodiment, multiple level sensors (e.g. top, middle, bottom) will be utilized to balance drainage and return as well as safety mechanisms An algorithm will be implemented into the console to control the drainage and the return of blood. If the top level fluid sensor is activated, the console will stop the drainage and increase the return of the blood. If the middle level fluid sensor is triggered, then the console will balance the drainage and the return of blood. If only the bottom level fluid sensor is activated, the console will stop the return of the blood.

In other embodiments, the first blood vessel is a right femoral vein and the second blood vessel is a left femoral vein. In other embodiments, the second blood vessel may be in a different location in the first blood vessel. In some embodiments, the first tube (120) has a length between about 140 cm to 170 cm. In other embodiments, the second tube (140) has a length between about 70 to 100 cm. In preferred embodiments, the first tube (120) may reach the pulmonary artery via the first blood vessel. In other embodiments, the second tube (140) may reach the inferior or superior vena cava.

The specific placement of the first tube and the second tube in the pulmonary artery and the vena cava, respectively, works to minimize hemodynamic compromise to the patient. Without wishing to limit the present invention to any theory or mechanism, there are two competing systems in the device: negative and positive pressures. The negative pressure is applied to suction the unwanted material and the positive pressure to deliver blood back to the patient. The opposing pressure negates the procedural effect of the device to the patient's hemodynamics. In preferred embodiments, the first filter (132) has a pore size of about 180 μm, and the second filter (134) has a pore size of about 40 μm. In some embodiments, a shape of the second filter (134) is configured to create laminar flow of blood from the reservoir (130) to the second tube (140). In preferred embodiments, the second filter (134) is conical in shape. In further embodiments, the top chamber (131) is transparent to visualize the unwanted materials and is configured to be opened for sample collection and subsequent pathology. In preferred embodiments, a negative pressure is constantly added to the reservoir to assist in drainage through the filter. In some embodiments, the device (100) further comprises a daughter catheter that is deployed from inside the first tube (120) to reach unwanted materials in smaller vessels. In other embodiments, the device further comprises a screw-in lock cap disposed at the first end of the first tube (120). In further embodiments, a disk is configured to be deployed through the screw-in lock cap to extract the unwanted material toward the first tube (120). In yet another embodiment, the disk is configured to be expanded when needed and in multiple configurations as desired by a user.

In some embodiments, the present invention features a method for removing unwanted material from a blood vessel using any of the devices described herein. The method may comprise: connecting the first end (122) of the first tube (120) to the first blood vessel, connecting the second end (144) of the second tube (140) to the second blood vessel, connecting the device (100) to a console (200), a vacuum pump, and a roller pump, using the console (200) to apply a first pressure on the first tube (120) with the vacuum pump, thereby suctioning blood from the blood vessel to the first tube (120), filtering the blood through the reservoir (130), wherein the unwanted material is filtered from the blood, using the console (200) to apply a second pressure at the bottom of the reservoir (130) using the roller pump, facilitating laminar flow of blood from the reservoir to the second tube (140), and returning the blood to the patient through the second tube (140).

In further embodiments, the console may apply artificial intelligence (AI) to control the first pressure and the second pressure or a flow rate of the device. In other embodiments, the console may use patient data to build safety measures of the device. Non-limiting examples of the safety measures include if MAP drops 10-30 mmHg or is below 65 mmHg, suction stops, and device delivers fluid/blood slowly until recovery or any number of options to improve safety. Other examples of the use of AI, include but are not limited to, inputting methodology to control volume by looking at RV strain, being slower with volume removal, or to deliver positive fluid balance to reduce the risk of dramatically lowering preload on the left side. In some embodiments the console may have sensors that can stop the device. Non-limiting examples of sensors include pressure sensors, flow sensors, bubble sensors, or level sensors.

Addition of integration with patient monitoring pressures. Determination of the speed of MAP drop to increase outflow before increasing inflow. Thus, if MAP drops 10 mmHg in less than 5 seconds it will reduce drainage and increase return until the MAP increases. Once the bottom level sensor is activated or detected with no volume, it completely stops the return and allows the clinician to decide how to proceed with additional volume or chemical pressure support. It can output a warning as to significant drops in MAP while adjusting the inflow/outflow rates. This will add a layer of safety in addition to making it simpler for the end user.

In some embodiments, the present invention features a device (100) for extracting an embolism from a blood vessel. In some embodiments, the device may comprise a first catheter (120), a reservoir (130), and a second catheter (140). The first catheter (120) may comprise a first end (122) that is configured to be disposed in a first blood vessel and a second end (124) that is connected to the reservoir (130). The reservoir (130) may have a filter disposed therein, and the reservoir may be connected to a vacuum pump and a roller pump. In further embodiments, the reservoir may have an outlet (137) disposed at a bottom end. In some embodiments, the second catheter (140) comprises a first end (142) that is connected to the outlet (137), and the second end (144) is configured to be disposed in a second blood vessel.

Without wishing to limit the present invention to any theory or mechanism, when suction is applied via the vacuum pump, blood containing the embolism is suctioned from the first blood vessel through the first catheter (120) to the reservoir (130). The blood is then filtered through the filter in the reservoir (130) to remove the embolism and air bubbles that may be present in the blood, and the blood is returned to the patient through the second catheter (140).

In some embodiments, the device (100) further comprises a console (200) that regulates the pressure of the vacuum pump and the roller pump. In other embodiments, the console (200) regulates a flowrate of blood being suctioned from a patient and a flowrate of blood being returned to a patient. In other embodiments, the console further comprises a pedal (210) to control the pressure or flowrate of the device. The vacuum pump may suction out blood from a patient using a negative pressure between about −150 mmHg to −700 mmHg. In preferred embodiments, the pressure used to suction out the blood may be staggered. As a non-limiting example, blood may be suctioned from a patient at about −700 mmHg to engage the embolism. Once the unwanted material is suctioned out with the blood, the pressure may be decreased to about 200 mmHg. In further embodiments, the console (200) comprises a sensor that controls the pressure applied to suction out blood.

In further embodiments, during suction of the blood from the patient, the reservoir (130) may be opened to visualize the blood. Without wishing to limit the present invention to any theory or mechanism, having a top filter in the reservoir (130) allows an operator of the device to open the reservoir (130) to see if the unwanted material has been filtered out and to send out for pathology. In some embodiments, the device (100) further comprises a daughter catheter that is deployed from inside the first catheter (120) to reach unwanted materials in smaller vessels. In other embodiments, the device further comprises a screw-in lock cap disposed at the first end of the first catheter (120). In further embodiments, a disk is configured to be deployed through the screw-in lock capto extract the unwanted material toward the first catheter (120). In yet another embodiment, the disk is configured to be expanded when needed and in multiple configurations as desired by a user.

In some embodiments, a roller head pump is attached to a bottom end of the reservoir (130). Without wishing to limit the present invention to any theory or mechanism, the roller head pump prevents blood from being suctioned in the opposite direction in the reservoir (130). The vacuum pump and the roller head pump create competing pressures that allow for the flow of blood in one direction and to prevent hemodynamic compromise in the patient.

In some embodiments, blood may be aspirated from a patient in a volume of up to 1.5 L. In other embodiments, the flowrate of blood being suctioned from a patient may be between about 200 mL/min to 3 L/min. In yet another embodiment, the flowrate of blood being returned to a patient may be between about 200 m L/min to 3 L/min. In some embodiments, the flowrate of the blood being suctioned out of the patient may not be the same as the flowrate of blood going back into the patient.

In other embodiments, the first blood vessel is a right femoral vein and the second blood vessel is a left femoral vein. In some embodiments, the second blood vessel is in a different location in the first blood vessel. In some embodiments, the first catheter (120) has a length between about 140 cm to 170 cm. In other embodiments, the second catheter (140) has a length between about 70 to 100 cm. In preferred embodiments, the first catheter (120) may reach the pulmonary artery. In other embodiments, the second catheter (140) may reach the inferior or superior vena cava. In preferred embodiments, the first filter (132) has a pore size of about 180 μm, and the first filter (134) has a pore size of about 40 μm. In other embodiments, the first filter (132) and the second filter (134) are conical in shape. Without wishing to limit the present invention to any theory or mechanism, the shape of the filters may be important to maintain laminar flow of the blood to eliminate air bubbles that may be in the blood and to minimize hemorrhage and damage.

In further embodiments, the device may further comprise an inlet disposed on a top end of the reservoir (130). Without wishing to limit the present invention to any theory or mechanism, the inlet may be used to administer a therapeutic composition, crystalloid, or additional blood products to the patient. As a non-limiting example, a spike line may be connected to a port or an inlet on the reservoir, and any necessary therapeutic compositions may be added through the spike line. Examples of therapeutic compositions may include, but are not limited to, anticoagulants, albumin, steroids, vasopressors, or electrolytes.

In other embodiments, the present invention features a method for removing unwanted material from a blood vessel using any of the devices described herein. The method may comprise: connecting the first end (122) of the first catheter (120) to the first blood vessel, connecting the second end (144) of the second catheter (140) to the second blood vessel, connecting the device (100) to a console (200), a vacuum pump, and a roller pump, using the console (200) to apply a first pressure on the first catheter (120) with the vacuum pump, thereby suctioning blood from the blood vessel to the first catheter (120), filtering the blood through the reservoir (130), wherein the unwanted material is filtered from the blood, using the console (200) to apply a second pressure at the bottom of the reservoir (130) using the roller pump, facilitating laminar flow of blood from the reservoir to the second catheter (140), and returning the blood to the patient through the second catheter (140).

In some embodiments, artificial intelligence (AI) may be integrated with the console to control the drainage of blood and to return the blood safely back to the patient. In this embodiment, patient data may be fed to the console to build other safety measures beyond basic level sensors or flow sensors. Non-limiting examples of the safety measures include that if MAP drops by 10 mmHg, suction stops and the device delivers fluid/blood slowly until recovery or any number of options to improve safety. Other examples of the use of AI, include but are not limited to, inputting methodology to control volume by looking at RV strain, being slower with volume removal, or to deliver positive fluid balance to reduce the risk of dramatically lowering preload on the left side. In some embodiments the console may be configured to stop the device based on input from one or more sensors. Non-limiting examples of sensors include pressure sensors, flow sensors, bubble sensors or level sensors.

In another embodiment, the present invention features a method for removing unwanted material from a heart or blood vessel. The method may comprise: connecting a first end (122) of a first catheter (120) to a first blood vessel and a second end (124) of the first catheter to a reservoir (130); connecting a second end (144) of a second catheter (140) to a second blood vessel and a first end (142) of the second catheter to the reservoir (130); applying a first pressure on the first catheter (120) via a first pump, thereby suctioning unwanted materials and blood from the heart or blood vessel to the first catheter (120); filtering the blood through the reservoir (130), where the unwanted material is filtered from the blood; applying a second pressure at the bottom of the reservoir (130) using a second pump, thereby facilitating laminar flow of blood from the reservoir to the second catheter (140); and returning the blood to the patient through the second catheter.

In additional embodiments, any of the devices described herein may be used to rapidly transfuse blood or other body fluids to a patient. In this configuration, transfusions can be rapidly performed because the return catheter can be placed near the heart or at the local area where the transfusion needs to take place. Current transfusions require IV, which limits the rate of transfusion and is not close to the heart. In other embodiments, the device may be connected to an oxygenator.

In some embodiments, the present invention features a device (100) for extracting unwanted material from a blood vessel. The device may include a suction catheter with a proximal end and a distal end. The distal end may be configured to be disposed in a first blood vessel. The suction catheter may be branched, or may have a single lumen through the catheter. An exterior diameter of the catheter at the distal end may be small enough to fit within the desired blood vessel. Alternatively, for example if the catheter is too big to fit within the desired blood vessel, a smaller daughter catheter may be passed through the suction catheter to fit within the desired blood vessel. The suction catheter may be attached to one or more pumps so as to generate a negative pressure for suctioning unwanted materials and blood from the blood vessel. In preferred embodiments, the entire lumen of the suction catheter has a large enough diameter to suction the unwanted material from the blood vessel without clogging the catheter. Furthermore, in branched suction catheters with the suction port disposed to the side of the catheter, the sweeping path may be at an angle designed to allow suctioning of the unwanted material from the blood vessel without clogging the catheter.

The device may include a return catheter comprising a proximal end and a distal end. The distal end may be configured to be disposed in a second blood vessel, for example, to return filtered blood suctioned from the first blood vessel to the second blood vessel. Just as the suction catheter may be branched or may have a single lumen through the catheter, the return catheter may be branched or may have a single lumen.

The device may include a flow system disposed between the suction catheter and the return catheter and fluidly coupled with the proximal ends of both catheters, wherein the flow system is configured to induce a suction flow from the suction catheter and a return flow to the return catheter. This flow system may include any suitable combination of pumps. As a non-limiting example, the flow system may include a suction pump, a fluidic connection with a vacuum system, and a return pump. In some embodiments, the suction pump and the return pump may each be roller pumps. In other embodiments, the flow system may include one or more manual pumps such as manually actuated syringes.

The device may also include a filter system disposed between the suction catheter and the return catheter and fluidly coupled with the proximal ends of both catheters, wherein the filter system is configured to capture unwanted material extracted from the blood vessel via the suction flow such that it is not returned to the second blood vessel via the return flow. As a non-limiting example, the filter system may include a filter reservoir having: a top chamber having a first filter disposed therein, the top chamber comprising an inlet which is fluidly coupled with the suction catheter; a bottom chamber comprising an outlet which is fluidly coupled with the return catheter, and a vacuum inlet fluidly coupled with a vacuum source; and a middle compartment disposed within the bottom chamber, the middle compartment having a second filter disposed therein. In some embodiments, the second filter comprises a funnel or cone-like structure. Without wishing to limit the present invention to any particular theory or mechanism, it is believed that the shape of the second filter may promote laminar flow, thereby reducing the possibility of introducing air bubbles into the return flow. In some embodiments, a pore size of the first filter is less than about 100, 110, 120, 130, 140, 150, 160, 170, 180, 190, 200, 220, 240, 260, 280, or 300 μm and a pore size of the second filter is less than about 10, 20, 30, 40, 50, 60, 70, 80, 90, or 100 μm.

According to preferred embodiments, the flow rates of the suction flow and the return flow may be independently adjusted. As a non-limiting example the suction and return pumps may operate at different speeds in order to provide a suction flow that is greater to, equal to, or less than the return flow. The device may additionally include a console configured to control pressure or flow rates of the suction flow and the return flow based on input from a plurality of sensors. Furthermore, the console may be configured to stop the suction flow, the return flow, or both if a potentially unsafe condition is predicted based on input from the plurality of sensors. As non-limiting examples, the plurality of sensors may include flow sensors, bubble sensors, pressure sensors, fluid level sensors, or a combination thereof.

In preferred embodiments, the device is configured to extract the unwanted material without significant overall loss of blood. As non-limiting examples, the device may be configured to extract the unwanted material with overall blood loss of less than about 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 60, 70, 80, 90, 100, 120, 140, 160, 180, 200, 250, 300, 350, 400, 450, 500, 600, 700, 800, 900, or 1000 cc of blood.

The flow system may induce a pulsed flow. As a non-limiting example, a flow rate or the speed of the operating pump may be increased and decreased cyclically to induce a vibration/sonication effect to assist in the extraction of unwanted materials. In some embodiments, the suction catheter and the return catheter each comprise a through-pathway configured for allowing implements to access through the catheter. As a non-limiting example, the through-pathway may provide for the application of the daughter cannula in the suction catheter in order to reach smaller spaces/cavities.

In some embodiments, the present invention features a suction catheter for extracting unwanted material from a blood vessel. As a non-limiting example, the catheter may include: a catheter body comprising a proximal end and a distal end, wherein the distal end is configured to be disposed in the blood vessel; a distal opening in the distal end of the catheter body; a suction port and an access port branching from the proximal end of the catheter body, wherein the suction port is configured to be fluidly coupled with a flow system configured to induce a suction flow from the suction catheter, and wherein the access port is configured to allow access through the catheter to the distal opening; and an air-lock coupled with the access port, wherein the air-lock is configured to seal itself or seal around an implement inserted through the access port. In preferred embodiments, a pathway from the distal opening to the suction port has a minimum diameter of at least about 1, 2, 3, 4, 5, 6, 7, 8, 9, 10, 11, 12, 13, 14, 15, 16, 17, 18, 19, or 20 mm. The implement may be a daughter catheter configured to be deployed through the catheter body to reach unwanted material from smaller blood vessels. Alternatively; the implement may be an expandable disk configured to be deployed through the catheter body to extract unwanted material toward the distal opening.

In some embodiments, the present invention may feature a method for removing unwanted material from a blood vessel. As a non-limiting example, the method may include: providing a device having a suction catheter, a return catheter, and flow and filter systems disposed between the suction catheter and the return catheter; inserting a distal end of the suction catheter into a first blood vessel; inserting a distal end of the return catheter into a second blood vessel; actuating the flow system so as to induce a suction flow from the suction catheter and a return flow to the return catheter; and using the filter system to filter blood removed from the first blood vessel via the suction flow prior to reintroducing it to the second blood vessel via the return catheter, thereby removing the unwanted material. In some embodiments, the first or second blood vessel may be a left or right femoral vein. As a non-limiting example, the first blood vessel may be a left femoral vein and the second blood vessel may be a right femoral vein.

### EXAMPLES

The following are non-limiting examples of the present invention. It is to be understood that said examples are not intended to limit the present invention in any way. Equivalents or substitutes are within the scope of the present invention.

### Example 1: A Suction Catheter of the Present Invention

The total length of the suction catheter is 145 cm. From the distal tip to the 30 cm mark, the catheter has variable diameters (24, 22, and 20 Fr). The thickness of the catheter is about 0.1 mm with a flexible spiral metal inside that is 3 mm apart per revolution. The distal tip of the catheter has 2 mm soft atraumatic soft material. After the 30 cm mark, the diameter is 24 Fr up to the locking mechanism. The thickness of the catheter is 0.1 mm with a flexible spiral metal insert that is 1 mm apart per revolution. The catheter has a screw-in locking mechanism that is 6 cm in length. The side port for negative suction or return is 9.5 mm. If steerability is needed a catheter with a rotating knob is also available.

There is a locking mechanism (air-lock) at the proximal end of the catheter: the cap screws into the catheter and as the cap is tightened, the rubber material inside the cap is compressed, thereby occluding the lumen. This mechanism prevents air from getting into the system during aspiration. At the end of the cap there is a rubber membrane that prevents blood from leaking.

The dilator of the catheter is 159 cm in length and tracks through a 0.35 inch standard stiff wire. The dilator is made of plastic and the distal tip of the dilator is tapered and smooth. It also has a tight fit with the distal segment of the catheter. The size of the dilator will depend on the distal catheter size. For DVT/small vessel clots, a 12 and 6 Fr size catheter can be used.

Description of the Daughter Catheter and Metal Connector

The length of the daughter catheter is 40 cm, and the catheter size can be 18, 16, or 14 Fr. The daughter catheter has a metal rod connector that is 126 cm. The distal tip of the catheter has a ball connector. The dilator length is 160 cm.

Description of the Disk:

The total length of the dilator is 166 cm and the disc catheter length is 182.7 cm. The disk length is 30 mm. The screw-in lock caplock mechanism is 4.6 cm long.

The disk is made out of 8 nitinol wires that are about 1 mm in thickness, 1 mm width×30 mm in length. When open, the total diameter of the disk is 18 mm. The 8 wires are attached distally to the dilator and proximally to a sliding catheter. The disk is connected to the said catheter at a 30-degree angle or vice versa (30-degree at the tip of the dilator). This angulation will then cause the disk to rotate and form a circular disk. During insertion to the body, that disk is retracted and is covered by a sheath. The first part of disk deployment is to pull the sheath to uncover the retracted disk. The second part is pushing the delivery catheter forward to the direction of the tip of the dilator in a circular fashion. This will be marked by 90, 180, 270, 360 degrees. The size of the disk is dependent on the forward position of the catheter. The size will also be marked relative to the degree of rotation. After the desired disk size is achieved, the locking mechanism can then be tightened to secure the disk size and the position.

Locking the catheter will prevent the disk from collapsing when the system is pulled for clot extraction. The disc with captured clots will be pulled toward the suction (long) catheter.

Description of the Return Cannula:

The total length of the return cannula is 35 to 70 cm, from the proximal end to the distal end. The diameter of the return cannula can be 19 Fr, 17 Fr, or 15 Fr. The tip has a 3 mm flexible/soft thin material, followed by 4 sets of 3 laser cut holes (12 holes total), each separated by 1 cm. The hole diameter is about 3 mm. From the tip to the first hole is 7 mm long. From the first hole to the suture lock is about 21 cm that will be reinforced with mesh metal just like the return cannula. From the suture lock to detail A is about 4 cm of just clear plastic for clamping. The screw-in lock cap is exactly as described in the long suction cannula. The dilator is 47 to 77 cm long and will be locked down with the screw-in lock cap (detail B) just as described in the long suction cannula.

Description of the Reservoir

The reservoir has plastic connectors at the base for blood delivery, and at the top for negative suction of blood and clots (these are 9.5 mm connectors). The suction connector is connected to the thrombectomy device and the delivery connector is connected to the return cannula using a 9.5 mm medical grade (e.g. Tygon) tube. The top-hat-shaped canister houses the top filter which is an inverted V that is 9.2 cm in height and is covered with 180-micron filter media to separate the aspirated thrombus from the blood. There are also two standard Luer lock connectors for delivering blood products and crystalloids to the system. In production, this top hat canister is screwed air tight into the main reservoir. A rubber O ring will ensure a complete seal between the top hat component and the main reservoir. There are two connectors at the bottom part of the top hat. One is for connection to the suction regulator and the other is a standard Luer lock. The reservoir is connected to the suction regulator using medical grade tubing. Inside the main reservoir is the main filter. The main filter comprises an outer circular plastic component with 4 support ribs for structural stability. A 40 micron filter is sandwiched between this structure and the v-shaped inside funnel. The V-shape funnel assists with laminar blood flow and prevents microbubble formation. A spill plate is at the bottom of this structure for support and to promote laminar blood flow to the bottom processed blood containment section. This section acts as a de-airing chamber and can accommodate 270 cc of volume. This section will be full of saline during device setup since the reservoir will be primed with 500 cc of normal saline.

This blood containment section also has multiple level sensors. The top sensor is at the 800 mL mark and the bottom is at the 100 mL. When the device is turned on to suction blood, the device will first give a predetermined amount (e.g. 200 cc) of fluid to the patient before the negative pressure is turned on. This increase in additional volume to the patient will increase preload and will mitigate the possible hemodynamic decline as the device aspirates blood from the pulmonary artery. Once fluid volume is achieved the suction will be turned on. The flow of blood in the negative suction catheter will be equal or close to the positive flow of blood to the return cannula which is blood going back to the patient. The device will stop when the amount of blood is only 100 mL.

The bottom of the reservoir has a 9.5 mm connector with three barbs. This is connected to the return cannula using a 9.5 mm medical grade tube. The entire assembly will be approximately 38.4 cm.

Description of the Console:

The console will generate negative force to suction the foreign materials along with blood from the patient into a reservoir; where blood and foreign materials are filtered and separated so that blood from the reservoir can be pumped back to the patient through a peristaltic pump with 9.5 mm″ medical grade tubing, multiple level sensors, bubble sensors, pressure sensors and flow sensors.

From the long suction cannula, a 9.5 mm inch medical grade tubing will be connected at the 9.5 mm sideport of the cannula to the top of the reservoir. Once the long cannula is situated appropriately at the clot, a peristaltic inflow pump will begin generating negative force with an RPM rate of 30 Mechanical suction at −20 mmHg will be applied to the reservoir to avoid positive pressure and assist in filter drainage via a regulator to the hospital suction source. The outflow pump will match the RPMs of the inflow pump to immediately balance volume. The higher the RPMs of the inflow pump, the higher the suction flow. There will be (multiple) level sensors embedded into the console at the back of the reservoir. The lowest will be placed at approximately 100 mL to start the transfusion at 30 RPM. If the lowest sensor is activated, it will stop the forward pump. The highest level will be at 800 mL to increase the RPM by 100% (2+LPM) but also stop the suction roller if it gets activated. Middle level sensors will balance flow appropriately. A drainage flow sensor and return flow sensor may also be present. The console may apply AI to match the flow rate. The console will also have a bubble sensor prior to the outflow pump head, acting as a safety mechanism to stop the return flow. There will be a one way valve in the tubing between the reservoir and the pump head to minimize gaseous micro-embolism due to high vacuum and cavitation. The peristaltic pump can be disengaged within the console or foot pedal and vacuum can be applied up to −700 mmHg from a hospital source. The peristaltic pump can be engaged to stop the vacuum source and stop all flow.

There will be a pressure sensor with flow sensor integrated distal to the pump head to match the flow rate of the drainage but also act as a safety mechanism to stop the pump head if post pressure is above 350 mmHg.

The vacuum source is from the hospital wall, which can be supplied up to −700 mmHg. The console can act as a suction device using mechanical suction if the tubing is not loaded into the inflow pump side. There will be a regulator inside the console that will interface with the lowest sensor and will be controlled automatically or manually by the end user and with a joystick at the field. The pump can be engaged or disengaged via the console or foot pedal to allow for vacuum to be applied to the line.

The console will be turned on and off with the pedal with an OFF override button on the pedal. Safety mechanisms that will stop the pump include bubble sensor, pressure sensors and level detectors.

Using the Device

Access is via the right and left femoral vein. The suction cannula is located on the right and the return cannula will be on the left. Access will be done via ultrasound guidance and a micropuncture 6 Fr system. A stiff 0.35 inch wire is then inserted to the 6 Fr sheath. The right 6 Fr sheath is then replaced with a 24 Fr sheath. The said sheath is sutured in place. Heparin should be given at 100 ug per kg. The left sheath is replaced over a 0.35 inch wire with the return cannula. A right heart catheter is then inserted inside the return cannula and is floated to the pulmonary artery. Over a 0.35 inch×280 cm in length wire the right heart catheter is removed and replaced with a 6 Fr pigtail catheter. The pigtail is then connected to a manifold or assist device for continuous pressure measurement and for angiography of the respected pulmonary artery. ACT should be checked after 5 mins of administration and should be >300. The pulmonary artery with the biggest clot burden should be treated first. A right heart catheter is inserted to the 24 Fr sheath and is floated to the target pulmonary artery. A 0.35 curve hydrophilic wire is inserted to the right heart catheter while the balloon is inflated, and the pulmonary artery is wired. After proper wire position is achieved distally, the right heart catheter is inserted distally for wire exchange. The curve hydrophilic wire is removed and a 1 cm stiff Amplatz guidewire is inserted to the target artery. The suction catheter and the return catheter are then connected to the console using the 9.5 mm tubing.

The console houses the reservoir for blood filtering and delivery. The reservoir is connected to the negative pressure regulator in the console. The negative pressure regulator is then connected to the hospital negative pressure wall system. The reservoir is then connected with a peristaltic pump to the thrombectomy device (suction catheter) using a 9.5 mm medical grade tube. Flow will be monitored using a flow sensor that is located just before the tube connects to the reservoir. The right heart catheter is removed over the wire and the desired thrombectomy catheter is inserted to the pulmonary artery. The negative pressure will start at a negative 100 mmHg or 30 RPMs and will increase to negative 600 mmHg or 150 RPMs if there is less than 100 mL/min of blood flow. If blood flow exceeds 500 mL/min then negative pressure will decrease to 100 mmHg or 10 RPMs. A peristaltic pump will deliver blood, fluids or medications back to the patient while suctioning and filtering blood.

### Example 2: Manual Extraction of Unwanted Materials and Return of Filtered Blood

In an embodiment without a console, the present invention may feature a manual device for removing unwanted material from a heart or blood vessel. The method for using said device may comprise: disposing a first end (122) of a first catheter (120) within a first blood vessel and connecting a second end (124) of the first catheter to a first duckbill-like one-way valve via a 9.5 mm diameter medical grade tubing. A tubing clamp will be present prior to the first one-way valve to clamp the tubing line to prevent bleeding or air. The first one-way valve will then be connected to a T-junction connector then to the second one-way valve and subsequently to a filter. The filter is then connected to the second end (144) of a second catheter (140) to a second blood vessel so that extracted blood can be returned to the patient. Another tubing clamp will be present between the filter and the second catheter to stop bleeding or transfusing any unwanted materials or allowing filter exchange. The filter apparatus/manifold is consisted of a 9.5 mm inlet at the top side, middle, or bottom; and a 9.5 mm outlet at the bottom side with a 40-120 micron filter in multiple potential configurations to filter out foreign materials; and a luer port at the very top to de-air or prime with crystalloid; and can hold 300-500 mL of blood. A 100 mL or larger lockable syringe may be quick-connected to the T-junction with the presence of another tubing clamp on the tubing between the syringe and the T-junction. These one-way valves will direct blood flow from the suction cannula to the filter then back to the return cannula through the pull and push of the syringe. The filter can be configured to fill from the bottom and flow through a step to the filter and back down to a bottom outlet, or the filter can fill from the top portion and pass through a filter to exit out of the bottom in various shapes allowing it to rest on the operative field and reduce the likelihood of air embolism to the patient. All ports, connectors, and tubing are 9.5 mm or ⅜ inches in diameter.

In another manual extraction embodiment, the setup for extracting unwanted materials could be reconfigured to include in sequence of: 1) the extraction cannula followed by tubing w/tubing clamp, followed by the 2) first one-way valve, which is connected to the 3) filter apparatus via tubing, which then connected to the T-junction connector that has the 100 ml syringe; then connected to the second one-way valve which is then connected the return cannula with a tubing clamp in the middle.

Using the Manual Extraction Device

As a non-limiting example of use of the extraction device, the clinician places the suction cannula in the heart or pulmonary artery as described. The syringe is filled with 100 mL of normal saline and used to de-air the tubing before connecting it to the return cannula. Once the entire apparatus is de-aired or primed with crystalloid, The clinician will close the tubing clamp #1 and #3 in anticipation of hooking up to the extraction and return cannula. The clinician will engage a clot, wet-to-wet connect the cannula and keep the tubing clamp closed. The 100 mL syringe will be pulled to generate negative pressure and lock it in place. If more negative is desired, the clinician can close the tubing clamp #2, which is right distal to the syringe and before the T-Junction connection; disconnect the syringe to reload it then quick-connect it back to the T-junction to pull again to generate more negative pressure. This step can be repeated as desired by the clinician. Once a negative suction is achieved within the syringe and the system, clinician will release tubing clamp #1 and #2. Once the syringe is filled, it can be pressed to reinfuse the blood because the one way valves will ensure proper flow from the suction cannula through the filter to the return cannula. This approach can be repeated until all or most unwanted materials are removed. The clinician can also choose to use a sheath or any available cannula (in lieu of the return cannula) within their practice that's deemed appropriate to infuse blood or crystalloid. If the filter is fully clotted; it can be exchanged through existing connections. After the completion of the procedure, normal saline can again be loaded into the syringe to displace the residual blood in the circuit so that blood can be given back to the patient.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Enumerated Embodiments: The following are non-limiting examples of enumerated embodiments.

Enumerated Embodiment 1: A device (100) for extracting unwanted material from a blood vessel, the device comprising:
a. a suction catheter comprising a proximal end and a distal end, wherein the distal end is configured to be disposed in a first blood vessel;
b. a return catheter comprising a proximal end and a distal end, therein the distal end is configured to be disposed in a second blood vessel;
c. a flow system disposed between the suction catheter and the return catheter and fluidly coupled with the proximal ends of both catheters, wherein the flow system is configured to induce a suction flow from the suction catheter and a return flow to the return catheter; and
d. a filter system disposed between the suction catheter and the return catheter and fluidly coupled with the proximal ends of both catheters, wherein the filter system is configured to capture unwanted material extracted from the blood vessel via the suction flow such that it is not returned to the second blood vessel via the return flow.

Enumerated Embodiment 2: The device of enumerated embodiment 1, wherein the flow system comprises a suction pump, a fluidic connection with a vacuum system, and a return pump.

Enumerated Embodiment 3: The device of enumerated embodiment 2, wherein the suction pump and the return pump each comprise roller pumps.

Enumerated Embodiment 4: The device of enumerated embodiment 1, wherein the flow system comprises a manually actuated syringe.

Enumerated Embodiment 5: The device of enumerated embodiment 1, wherein the filter system comprises a filter reservoir comprising:
a. a top chamber having a first filter disposed therein, the top chamber comprising an inlet which is fluidly coupled with the suction catheter;
b. a bottom chamber comprising an outlet which is fluidly coupled with the return catheter, and a vacuum inlet fluidly coupled with a vacuum source; and
c. a middle compartment disposed within the bottom chamber, the middle compartment having a second filter disposed therein.

Enumerated Embodiment 6: The device of enumerated embodiment 5, wherein the second filter comprises a funnel or cone-like structure.

Enumerated Embodiment 7: The device of enumerated embodiment 5, wherein a pore size of the first filter is about 180 μm and a pore size of the second filter is about 40 μm.

Enumerated Embodiment 8: The device of enumerated embodiment 1, wherein the flow rates of the suction flow and the return flow may be independently adjusted.

Enumerated Embodiment 9: The device of enumerated embodiment 1, additionally comprising a console configured to control pressure or flow rates of the suction flow and the return flow based on input from a plurality of sensors.

Enumerated Embodiment 10: The device of enumerated embodiment 9, wherein the console is configured to stop the suction flow, the return flow, or both if a potentially unsafe condition is predicted based on input from the plurality of sensors.

Enumerated Embodiment 11: The device of enumerated embodiment 9, wherein the plurality of sensors comprise flow sensors, bubble sensors, pressure sensors, fluid level sensors, or a combination thereof.

Enumerated Embodiment 12: The device of enumerated embodiment 1, wherein the device is configured to extract the unwanted material without significant overall loss of blood.

Enumerated Embodiment 13: The device of enumerated embodiment 1, wherein the flow system is configured to induce a pulsed flow.

Enumerated Embodiment 14: The device of enumerated embodiment 1, wherein the suction catheter and the return catheter each comprise a through-pathway configured for allowing implements to access through the catheter.

Enumerated Embodiment 15: A suction catheter for extracting unwanted material from a blood vessel, the catheter comprising:
a. a catheter body comprising a proximal end and a distal end, wherein the distal end is configured to be disposed in the blood vessel;
b. a distal opening in the distal end of the catheter body;
c. a suction port and an access port branching from the proximal end of the catheter body, wherein the suction port is configured to be fluidly coupled with a flow system configured to induce a suction flow from the suction catheter, and wherein the access port is configured to allow access through the catheter to the distal opening; and
d. an air-lock coupled with the access port, wherein the air-lock is configured to seal itself or seal around an implement inserted through the access port.

Enumerated Embodiment 16: The catheter of enumerated embodiment 15, wherein a pathway from the distal opening to the suction port has a minimum diameter of at least about 9 mm.

Enumerated Embodiment 17: The catheter of enumerated embodiment 15, wherein the implement comprises a daughter catheter configured to be deployed through the catheter body to reach unwanted material from smaller blood vessels.

Enumerated Embodiment 18: The catheter of enumerated embodiment 15, wherein the implement comprises an expandable disk configured to be deployed through the catheter body to extract unwanted material toward the distal opening.

Enumerated Embodiment 19: A method for removing unwanted material from a blood vessel, the method comprising:
a. providing a device comprising: a suction catheter; a return catheter; a flow system disposed between the suction catheter and the return catheter; and a filter system disposed between the suction catheter and the return catheter;
b. inserting a distal end of the suction catheter into a first blood vessel;
c. inserting a distal end of the return catheter into a second blood vessel;
d. actuating the flow system so as to induce a suction flow from the suction catheter and a return flow to the return catheter; and
e. using the filter system to filter blood removed from the first blood vessel via the suction flow prior to reintroducing it to the second blood vessel via the return catheter, thereby removing the unwanted material.

Enumerated Embodiment 20: The method of enumerated embodiment 19, wherein the first or second blood vessel comprises a left or right femoral vein.

Enumerated Embodiment 21: A device (100) for extracting unwanted material from a blood vessel, the device comprising:
a. a first tube (120) comprising a first end (122) and a second end (124), wherein the first end (122) is configured to be disposed in a first blood vessel and the second end (124) is connected to a reservoir (130);
b. the reservoir (130) comprising: a top chamber (131) having a first filter (132) disposed therein; a bottom chamber (136) comprising an outlet (137), wherein a vacuum inlet (135) connects the bottom chamber middle chamber (1363) to a vacuum source, and wherein the bottom chamber is connected to a roller pump; and a middle compartment (133) disposed within the bottom chamber (136), the middle compartment (133) having a second filter (134) disposed therein; and
c. a second tube (140) comprising a first end (142) and a second end (144), wherein the first end (142) is connected to the outlet (137) of the reservoir (130) and the second end (144) is configured to be disposed in a second blood vessel.

Enumerated Embodiment 22: The device (100) of enumerated embodiment 21, wherein the top chamber is removable.

Enumerated Embodiment 23: The device (100) of enumerated embodiment 21, wherein the top chamber has multiple access ports.

Enumerated Embodiment 24: The device (100) of enumerated embodiment 21, wherein the second filter (134) comprises a funnel or cone-like structure.

Enumerated Embodiment 25: The device (100) of enumerated embodiment 21, wherein the vacuum or peristaltic pump controls flow of blood out of a patient.

Enumerated Embodiment 26: The device (100) of enumerated embodiment 21, wherein the roller pump controls flow of blood into a patient.

Enumerated Embodiment 27: The device (100) of enumerated embodiment 21, wherein the device (100) further comprises a console (200) to control pressure or flow rate of the device, and level sensors to control the volume of blood in the reservoir (130).

Enumerated Embodiment 28: The device (100) of enumerated embodiment 27, wherein the console (200) further comprises a pedal (210) to control the pressure or flow rate of the device.

Enumerated Embodiment 29: The device (100) of enumerated embodiment 27 wherein the pressure is between about 0 mmHg to −700 mmHg.

Enumerated Embodiment 30: The device (100) of enumerated embodiment 21, wherein the first blood vessel is a right or left femoral vein.

Enumerated Embodiment 31: The device (100) of enumerated embodiment 2 wherein the second blood vessel is a right or left femoral vein.

Enumerated Embodiment 32: The device (100) of enumerated embodiment 21, wherein the unwanted material is a foreign body, a clot, or other infectious material.

Enumerated Embodiment 33: The device (100) of enumerated embodiment 21, wherein the first tube (120) has a length between about 140 cm to 170 cm.

Enumerated Embodiment 34: The device (100) of enumerated embodiment 21, wherein a shape of the second filter (134) is configured to create laminar flow of blood from the reservoir (130) to the second tube (140).

Enumerated Embodiment 35: The device (100) of enumerated embodiment 34, wherein the second filter (134) is conical in shape.

Enumerated Embodiment 36: The device (100) of enumerated embodiment 21, wherein a pore size of the first filter (132) is about 180 μm.

Enumerated Embodiment 37: The device (100) of enumerated embodiment 21, wherein a pore size of the second filter (134) is about 40 μm.

Enumerated Embodiment 38: The device (100) of enumerated embodiment 21, wherein the top chamber (131) is transparent and is configured to be opened during or post operation of the device (100).

Enumerated Embodiment 39: The device (100) of enumerated embodiment 21, wherein the top chamber (131) is accessible to visualize and to collect the filtered unwanted material.

Enumerated Embodiment 40: The device (100) of enumerated embodiment 21, wherein the device further comprises a daughter catheter that is deployed from inside the first tube (120) to reach unwanted materials in smaller vessels.

Enumerated Embodiment 41: The device (100) of enumerated embodiment 21, wherein the device further comprises a screw-in lock cap disposed at the first end of the first tube (120).

Enumerated Embodiment 42: The device (100) of enumerated embodiment 41, wherein a disk is configured to be deployed through the screw-in lock capto extract the unwanted material toward the first tube (120).

Enumerated Embodiment 43: The device (100) of enumerated embodiment 42, wherein the disk is configured to be expanded when needed and in multiple configurations as desired by a user.

Enumerated Embodiment 44: A device (100) for extracting an embolism from a blood vessel, the device comprising:
a. a first catheter (120) comprising a first end (122) and a second end (124), wherein the first end (122) is configured to be disposed in a first blood vessel and the second end (124) is connected to a reservoir (130);
b. the reservoir (130) having a filter disposed therein, wherein the reservoir (130) is connected to a vacuum pump and a roller pump, wherein an outlet (137) is disposed at a bottom end of the reservoir (130); and
c. a second catheter (140) comprising a first end (142) and a second end (144), wherein the first end (142) is connected to the outlet (137) of the reservoir (130) and the second end (144) is configured to be disposed in a second blood vessel.

Enumerated Embodiment 45: The device of enumerated embodiment 44, wherein the device (100) further comprises a console (200).

Enumerated Embodiment 46: The device of enumerated embodiment 45 wherein the console (200) regulates a pressure of the vacuum pump, and engagement of the roller pump.

Enumerated Embodiment 47: The device of enumerated embodiment 45, wherein the console (200) regulates a flowrate of blood being suctioned from a patient.

Enumerated Embodiment 48: The device of enumerated embodiment 45, wherein the console (200) regulates a flowrate of blood being returned to a patient.

Enumerated Embodiment 49: The device (100) of enumerated embodiment 45, wherein the console (200) further comprises a pedal (210) to control the pressure or flowrate of the device.

Enumerated Embodiment 50: The device (100) of enumerated embodiment 46, wherein the pressure is between about 0 mmHg to −700 mmHg.

Enumerated Embodiment 51: The device (100) of enumerated embodiment 44, wherein blood is aspirated from a patient in a volume up to about 1.5 L.

Enumerated Embodiment 52: The device (100) of enumerated embodiment 44, wherein a flowrate of the blood being suctioned from a patient is between about 200 mL/min to 3 L/min.

Enumerated Embodiment 53: The device (100) of enumerated embodiment 44, wherein a flowrate of the blood being returned to a patient is between about 200 mL/min to 3 L/min.

Enumerated Embodiment 54: The device (100) of enumerated embodiment 44, wherein a flow rate of blood being suctioned from a patient is different from a flow rate of blood being returned to the patient.

Enumerated Embodiment 55: The device (100) of enumerated embodiment 44, wherein the reservoir (130) further comprises a second inlet disposed at a top of the reservoir (130).

Enumerated Embodiment 56: The device (100) of enumerated embodiment 44, wherein a therapeutic composition, saline, or additional blood products is administered to available ports connected to the device (100).

Enumerated Embodiment 57: The device (100) of enumerated embodiment 44, wherein the device further comprises a daughter catheter that is deployed from inside the first catheter (120) to reach unwanted materials in smaller vessels.

Enumerated Embodiment 58: The device (100) of enumerated embodiment 44, wherein the device further comprises a screw-in lock cap disposed at the first end of the first catheter (120).

Enumerated Embodiment 59: The device (100) of enumerated embodiment 58, wherein a disk is configured to be deployed through the screw-in lock capto extract the unwanted material toward the first tube (120).

Enumerated Embodiment 60: The device (100) of enumerated embodiment 58, wherein the disk is configured to be expanded when needed and in multiple configurations as desired by a user.

Enumerated Embodiment 61: A method for removing unwanted material from a blood vessel using the device (100) of enumerated embodiment 21, the method comprising:
a. connecting the first end (122) of the first tube (120) to the first blood vessel;
b. connecting the second end (144) of the second tube (140) to the second blood vessel;
c. connecting the device (100) to a console (200), a vacuum pump, and two roller pumps;
d. using the console (200) to apply a first pressure on the first tube (120) with the vacuum pump, thereby suctioning unwanted materials and blood from the heart or blood vessel to the first tube (120);
e. filtering the blood through the reservoir (130), wherein the unwanted material is filtered from the blood;
f. using the console (200) to apply a second pressure at the bottom of the reservoir (130) using the roller pump, facilitating laminar flow of blood from the reservoir to the second tube (140); and
g. returning the blood to the patient through the second tube (140).

Enumerated Embodiment 62: The method of enumerated embodiment 61 wherein the console (200) applies AI to control the first pressure and second pressure.

Enumerated Embodiment 63: The method of enumerated embodiment 61, wherein the console (200) applies AI to control a flow rate of the device.

Enumerated Embodiment 64: The method of enumerated embodiment 61, wherein the console (200) uses patient data to build safety measures of the device.

Enumerated Embodiment 65: The method of enumerated embodiment 62, wherein the console will stop the drainage when a patient MAP drops below 65 mmHg or greater than 10 mmHg of baseline.

Enumerated Embodiment 66: A method for removing unwanted material from a blood vessel using the device (100) of enumerated embodiment 44, the method comprising:
a. connecting the first end (122) of the catheter (120) to the first blood vessel;
b. connecting the second end (144) of the second catheter (140) to the second blood vessel;
c. connecting the device (100) to a console (200);
d. using the console (200) to apply a first pressure on the first catheter (120) with the vacuum pump, thereby suctioning blood from the blood vessel to the first catheter (120);
e. filtering the blood through the reservoir (130), wherein the unwanted material is filtered from the blood
f. using the console (200) to apply a second pressure at the bottom of the reservoir (130) using the roller pump, facilitating laminar flow of blood from the reservoir to the second catheter (140); and
g. returning the blood to the patient through the second catheter (140).

Enumerated Embodiment 67: The method of enumerated embodiment 66, wherein the console (200) applies AI to control the first pressure and second pressure.

Enumerated Embodiment 68: The method of enumerated embodiment 66, wherein the console (200) applies AI to control a flow rate of the device.

Enumerated Embodiment 69: The method of enumerated embodiment 66, wherein the console (200) uses patient data to build safety measures of the device.

Enumerated Embodiment 70: The method of enumerated embodiment 66, wherein a daughter catheter can be deployed inside the first tube (120) to reach foreign materials in the smaller vessels.

Enumerated Embodiment 71: The method of enumerated embodiment 66, wherein a screw-in lock cap is disposed at the first end of the first catheter (120).

Enumerated Embodiment 72: The method of enumerated embodiment 71, wherein a disk is configured to be deployed through the screw-in lock capto extract unwanted material toward the first tube (120).

Enumerated Embodiment 73: The method of enumerated embodiment 71, wherein the disk is configured to be expanded when needed and in multiple configurations as desired by a user.

Enumerated Embodiment 74: A method for removing unwanted material from a heart or blood vessel, the method comprising:
a. connecting a first end (122) of a first catheter (120) to a first blood vessel and a second end (124) of the first catheter to a reservoir (130);
b. connecting a second end (144) of a second catheter (140) to a second blood vessel and a first end (142) of the second catheter to the reservoir (130);
c. applying a first pressure on the first catheter (120) via a first pump, thereby suctioning unwanted materials and blood from the heart or blood vessel to the first catheter (120);
d. filtering the blood through the reservoir (130), wherein the unwanted material is filtered from the blood;
e. applying a second pressure at the bottom of the reservoir (130) using a second pump, thereby facilitating laminar flow of blood from the reservoir to the second catheter (140); and
f. returning the blood to the patient through the second catheter (140).

The reference numbers recited in the below claims are solely for ease of examination of this patent application, and are exemplary, and are not intended in any way to limit the scope of the claims to the particular features having the corresponding reference numbers in the drawings.

What is claimed is:

1. A device (100) for extracting unwanted material from a blood vessel, the device comprising:
a. a suction catheter comprising a proximal end and a distal end, wherein the distal end is configured to be disposed in a first blood vessel;
b. a return catheter comprising a proximal end and a distal end, wherein the distal end is configured to be disposed in a second blood vessel;
c. a flow system disposed between the suction catheter and the return catheter and fluidly coupled with the proximal ends of both catheters, wherein the flow system is configured to induce a suction flow from the suction catheter and a return flow to the return catheter; and
d. a filter system disposed between the suction catheter and the return catheter and fluidly coupled with the proximal ends of both catheters, wherein the filter system is configured to capture unwanted material extracted from the blood vessel via the suction flow such that it is not returned to the second blood vessel via the return flow.

2. The device of claim 1, wherein the flow system comprises a suction pump, a fluidic connection with a vacuum system, and a return pump.

3. The device of claim 2, wherein the suction pump and the return pump each comprise roller pumps.

4. The device of claim 1, wherein the flow system comprises a manually actuated syringe.

5. The device of claim 1, wherein the filter system comprises a filter reservoir comprising:
a. a top chamber having a first filter disposed therein, the top chamber comprising an inlet which is fluidly coupled with the suction catheter;
b. a bottom chamber comprising an outlet which is fluidly coupled with the return catheter, and a vacuum inlet fluidly coupled with a vacuum source; and
c. a middle compartment disposed within the bottom chamber, the middle compartment having a second filter disposed therein.

6. The device of claim 5, wherein the second filter comprises a funnel or cone-like structure.

7. The device of claim 5, wherein a pore size of the first filter is about 180 μm and a pore size of the second filter is about 40 μm.

8. The device of claim 1, wherein the flow rates of the suction flow and the return flow may be independently adjusted.

9. The device of claim 1, additionally comprising a console configured to control pressure or flow rates of the suction flow and the return flow based on input from a plurality of sensors.

10. The device of claim 9, wherein the console is configured to stop the suction flow, the return flow, or both if a potentially unsafe condition is predicted based on input from the plurality of sensors.

11. The device of claim 9, wherein the plurality of sensors comprise flow sensors, bubble sensors, pressure sensors, fluid level sensors, or a combination thereof.

12. The device of claim 1, wherein the device is configured to extract the unwanted material without significant overall loss of blood.

13. The device of claim 1, wherein the flow system is configured to induce a pulsed flow.

14. The device of claim 1, wherein the suction catheter and the return catheter each comprise a through-pathway configured for allowing implements to access through the catheter.

15. A suction catheter for extracting unwanted material from a blood vessel, the catheter comprising:
a. a catheter body comprising a proximal end and a distal end, wherein the distal end is configured to be disposed in the blood vessel;
b. a distal opening in the distal end of the catheter body;
c. a suction port and an access port branching from the proximal end of the catheter body, wherein the suction port is configured to be fluidly coupled with a flow system configured to induce a suction flow from the suction catheter, and wherein the access port is configured to allow access through the catheter to the distal opening; and
d. an air-lock coupled with the access port, wherein the air-lock is configured to seal itself or seal around an implement inserted through the access port.

16. The catheter of claim 15, wherein a pathway from the distal opening to the suction port has a minimum diameter of at least about 9 mm.

17. The catheter of claim 15, wherein the implement comprises a daughter catheter configured to be deployed through the catheter body to reach unwanted material from smaller blood vessels.

18. The catheter of claim 15, wherein the implement comprises an expandable disk configured to be deployed through the catheter body to extract unwanted material toward the distal opening.

19. A method for removing unwanted material from a blood vessel, the method comprising:
a. providing a device comprising:
i. a suction catheter;
ii. a return catheter;
iii. a flow system disposed between the suction catheter and the return catheter; and
iv. a filter system disposed between the suction catheter and the return catheter;
b. inserting a distal end of the suction catheter into a first blood vessel;
c. inserting a distal end of the return catheter into a second blood vessel;
d. actuating the flow system so as to induce a suction flow from the suction catheter and a return flow to the return catheter; and
e. using the filter system to filter blood removed from the first blood vessel via the suction flow prior to reintroducing it to the second blood vessel via the return catheter, thereby removing the unwanted material.

20. The method of claim 19, wherein the first or second blood vessel comprises a left or right femoral vein.
